(19) United States
(12) Patent Application Publication
(10) Pub. No.: US 2022/0101997 A1
(43) Pub. Date: Mar. 31, 2022

(54) PROCESSING TIME-DOMAIN AND FREQUENCY-DOMAIN REPRESENTATIONS OF EEG DATA

(71) Applicant: X Development LLC, Mountain View, CA (US)

(72) Inventors: Asim Iqbal, Zurich (CH); Mustafa Ispir, Mountain View, CA (US); Garrett Raymond Honke, Mountain View, CA (US); Nina Thigpen, Sunnyvale, CA (US); Vladimir Miskovic, Binghamton, NY (US); Pramod Gupta, Mountain View, CA (US)

(21) Appl. No.: 17/039,303

(22) Filed: Sep. 30, 2020

(51) Int. Cl. G16H 50/20; G06N 3/08

(52) U.S. Cl. CPC G16H 50/20; G06N 3/08

(57) ABSTRACT

Methods, systems, and apparatus, including computer programs encoded on computer storage media, for processing representations of EEG measurements. One of the methods includes obtaining a plurality of EEG signal measurements corresponding to respective EEG trials of a user; generating a time-domain representation from the plurality of EEG signal measurements, where the time-domain representation includes a plurality of rows, and where each row corresponds to a different set of one or more EEG signal measurements; applying the time-domain representation as input to a neural network having a plurality of network parameters, final values of the network parameters having been determined by a transfer learning process where the neural network is initially trained to perform an image processing task and the neural network is subsequently trained to perform EEG analysis; and obtaining, from the neural network, a mental health prediction for the user.

### BACKGROUND

This specification relates to generating outputs using neural networks.

Neural networks are machine learning models that employ multiple layers of operations to predict one or more outputs from one or more inputs. Neural networks typically include one or more hidden layers situated between an input layer and an output layer. The output of each layer is used as input to another layer in the network, e.g., the next hidden layer or the output layer.

Each layer of a neural network specifies one or more transformation operations to be performed on input to the layer. Some neural network layers have operations that are referred to as neurons. Each neuron receives one or more inputs and generates an output that is received by another neural network layer. Often, each neuron receives inputs from other neurons, and each neuron provides an output to one or more other neurons.

An architecture of a neural network specifies what layers are included in the network and their properties, as well as how the neurons of each layer of the network are connected. In other words, the architecture specifies which layers provide their output as input to which other layers and how the output is provided.

The transformation operations of each layer are performed by computers having installed software that implement the transformation operations. Thus, a layer being described as performing operations means that the computers implementing the transformation operations of the layer perform the operations.

Each layer generates one or more outputs using the current values of a set of parameters for the layer. Training the neural network thus involves continually performing a forward pass on the input, computing gradient values, and updating the current values for the set of parameters for each layer using the computed gradient values. Once a neural network is trained, the final set of parameter values can be used to make predictions in a production system.

### SUMMARY

This specification describes a system that processes electroencephalogram (EEG) signal measurements to generate a mental health prediction of a user. In particular, the system can process one or more different image-based representations of the EEG signal measurements. For example, the system can obtain a two-dimensional time-domain representation of one or more EEG signal measurements, and process the time-domain representation using a first neural network to generate the mental health prediction for the user. Instead or in addition, the system can obtain one or more two-dimensional frequency-domain representations of the one or more EEG signal measurements, and process the frequency-domain representations using the first neural network and/or a second neural network to generate the mental health prediction for the user. Each of the one or more frequency-domain representations can correspond to a respective different range of frequencies.

In some implementations, the first neural network and/or the second neural network is a transfer-learned neural network. That is, a training system can obtain pre-trained parameters of the neural networks that have been trained to perform a different image processing task, and then use the pre-trained parameters to determine final parameters of the neural networks, e.g., by fine-tuning the pre-trained parameters using EEG training examples.

This specification also describes a system that determines optimal frequency ranges of frequency-domain representations for generating mental health predictions of users. That is, the system can determine one or more frequency ranges that, when used to process EEG signal measurements to generate frequency-domain representations, encode the most useful information into the frequency-domain representations. The system can then process the frequency-domain representations using a neural network to generate the mental health predictions. In particular, the system can treat the frequency ranges corresponding to the input of the neural network as a hyperparameter of the neural network, training multiple versions of the neural network that each correspond to a different frequency range in order to determine the optimal frequency range.

The subject matter described in this specification can be implemented in particular embodiments so as to realize one or more of the following advantages.

Time-domain representations and frequency-domain representations of EEG signal measurements can encode different information about the EEG signal measurements, and so using one type of representation can be more effective than another depending on the use case. Furthermore, in systems that leverage frequency-domain EEG representations, the optimal frequency range for generating frequency-domain representations can depend on the specific use case. Using techniques described in this specification, a system can determine the optimal frequency range for a particular use case.

In some cases, processing both time-domain representations and frequency-domain representations using respective subnetworks of the same neural network can extract more information about the EEG signal measurements than processing any single representation individually. By using an ensemble of multiple different neural networks corresponding to different representations of the EEG data, the system can generate mental health predictions that are more accurate than any one single neural network would generate. In particular, the time-domain and the frequency-domain representations can encode different information about the same EEG data, and so by processing both types of representations, the system can leverage more useful information to generate mental health predictions that are more accurate.

Leveraging a transfer-learned neural network can further allow the system to extract rich information from different representations of EEG signal measurements. A pre-trained neural network corresponding to an image processing task can be trained to extract high-level information from images in order to perform the image processing task, e.g., classifying object depicted in the image. This information can also be useful, when extracted from a time-domain or frequency-domain image representation, in predicting the mental health status of a user.

Furthermore, according to implementations of the present disclosure the pre-trained neural network is generally trained on a training data set of images that is larger than available datasets of EEG training data, e.g., a training data set that includes a hundred thousand, a million, ten million, or a hundred million images. A system can therefore more efficiently and effectively train a neural network by obtaining parameter values trained on the large training data set of images and fine-tuning the parameter values using a smaller EEG training data set, than if the system attempted to train the neural network on the smaller EEG training data set alone.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a diagram of an example EEG processing system.

FIG. 2A is an illustration of an example time-domain representation of EEG signal measurements.

FIG. 2B is an illustration of an example frequency-domain representation of an EEG signal measurement.

FIG. 3 is a diagram of an example training system.

FIG. 4 is a flow diagram of an example process for processing one or more EEG signal measurements to generate a mental health prediction for a user.

FIG. 5 is a flow diagram of an example process for determining the optimal frequency range of frequency-domain representations for generating mental health predictions for users.

Like reference numbers and designations in the various drawings indicate like elements.

### DETAILED DESCRIPTION

This specification describes a system that processes EEG signal measurements to generate a mental health prediction of a user.

In this specification, a mental health prediction of a user can be a prediction regarding any aspect of the mental health status of the user. For example, a mental health prediction can represent the likelihood that the user has one or more particular mental illnesses. As another example, a mental health prediction can represent a classification of a personality type of the user, e.g., a prediction that the user belongs to a particular one out of N possible personality types. As another example, the mental health prediction can represent a likelihood that the user will develop one or more illnesses or traits in the future, e.g., a likelihood that the user will develop a particular addiction. As another example, the mental health prediction can represent a prediction of a current mental state of the user, e.g., a prediction regarding attention allocation, anticipation, surprise, etc.

An EEG signal measurement characterizes brain activity of a user. To capture an EEG signal measurement, multiple electrodes are placed on the scalp of the user at different locations, and each electrode measures the electrical activity of the brain at the corresponding location over a period of time. The measurement captured by each electrode is a one-dimensional time series, where each element of the time series represents the amplitude of electrical activity of the brain at the corresponding location at a particular time point. The time series captured by each electrode can be included in a respective channel of the EEG signal measurement. That is, a given EEG signal measurement characterizes the brain activity of the user during a particular period of time and includes one or more channels that each corresponds to a respective location on the scalp of the user.

In some cases, EEG signal measurements are captured while the user is performing a cognitive-behavioral task, referred to in this specification as an “EEG task.” A system can present a prompt corresponding to the EEG task to the user, e.g., using a graphical user interface, and capture an EEG signal measurement in response to the prompt; the process of capturing an EEG signal measurement is referred to in this specification as an “EEG trial.” In some cases, the EEG task can be passive, e.g., the prompt can be to look at an image. In some other cases, the EEG task can be active, e.g., the prompt can be to select a choice from a set of options.

An EEG task can have one or more different prompt types that represent different categories of prompts of the EEG task. The brain activity of the user in response to prompts of different categories will generally be different. As a particular example, an EEG task can be to view an image, a first prompt type can be to view pleasant images, and a second prompt type can be to view unpleasant images. As another example, an EEG task can be to receive real or fake monetary rewards and punishments, e.g., in a gambling context; in this case, a first prompt type can be to view positive monetary reinforcement, and a second prompt type can be to view negative monetary reinforcement.

An EEG signal measurement captured in response to a prompt of a first prompt type and an EEG signal measurement captured in response to a prompt of a second prompt type can be compared in order to generate a mental health prediction of the user. For example, EEG signal measurements corresponding to different respective prompt types can be compared to diagnose one or more mental illnesses, e.g., major depressive disorder, bipolar disorder, or anxiety.

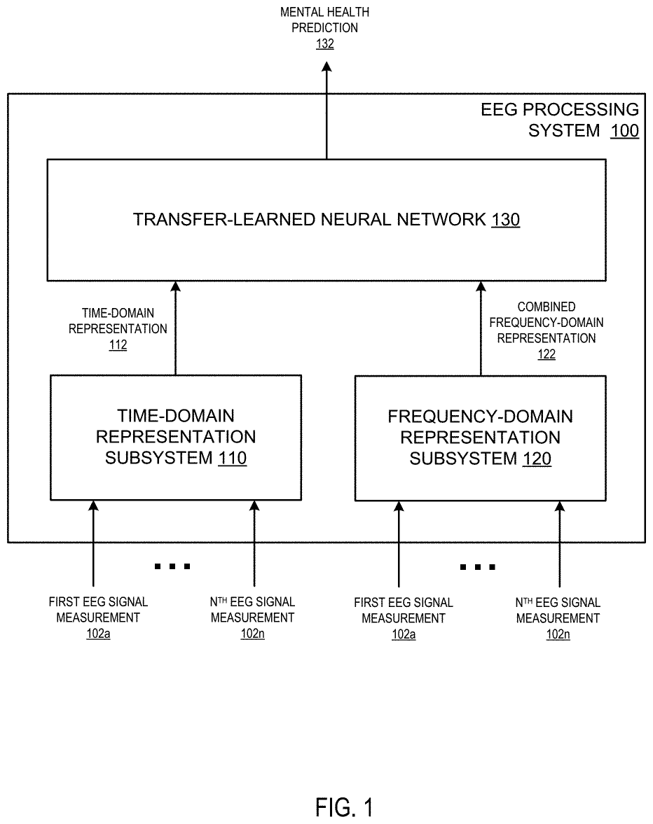

FIG. 1 is a diagram of an example EEG processing system 100. The EEG processing system 100 is an example of a system implemented as computer programs on one or more computers in one or more locations, in which the systems, components, and techniques described below can be implemented.

The EEG embedding system 100 receives as input N EEG signal measurements 102a-n corresponding to the same user, where N≥1. In some implementations, each of the EEG signal measurements 102a-n correspond to the same particular prompt type of an EEG task; that is, each EEG signal measurement 102a-n was captured during a respective EEG trial in which the user was presented a prompt of the particular prompt type of the EEG task.

The EEG processing system 100 processes the EEG signal measurements 102a-n to generate a mental health prediction 132 for the user.

Often, an EEG signal measurement corresponding to a single EEG trial can have a high degree of noise. That is, a single EEG signal measurement might not be an accurate representation of the response of the brain of the user to the corresponding prompt type of an EEG task. Therefore, in order to generate an accurate mental health prediction 132 of the user that represents the true mental health of the user, the EEG processing system 100 can process multiple different EEG signal measurements, e.g., multiple different EEG signal measurements corresponding to a particular prompt type that were gathered during respective different EEG trials. Thus, because the mental health prediction 132 reflects information from multiple different signal measurements gathered during independent EEG trials, the prediction 132 does not have as much noise as a single EEG signal measurement.

The EEG processing system 100 includes a time-domain representation subsystem 110, a frequency-domain representation subsystem 120, and a transfer-learned neural network 130.

The time-domain representation subsystem 110 obtains the EEG signal measurements 102a-n and processes the EEG signal measurements 102a-n to generate a time-domain representation 112 of the EEG signal measurements 102a-n.

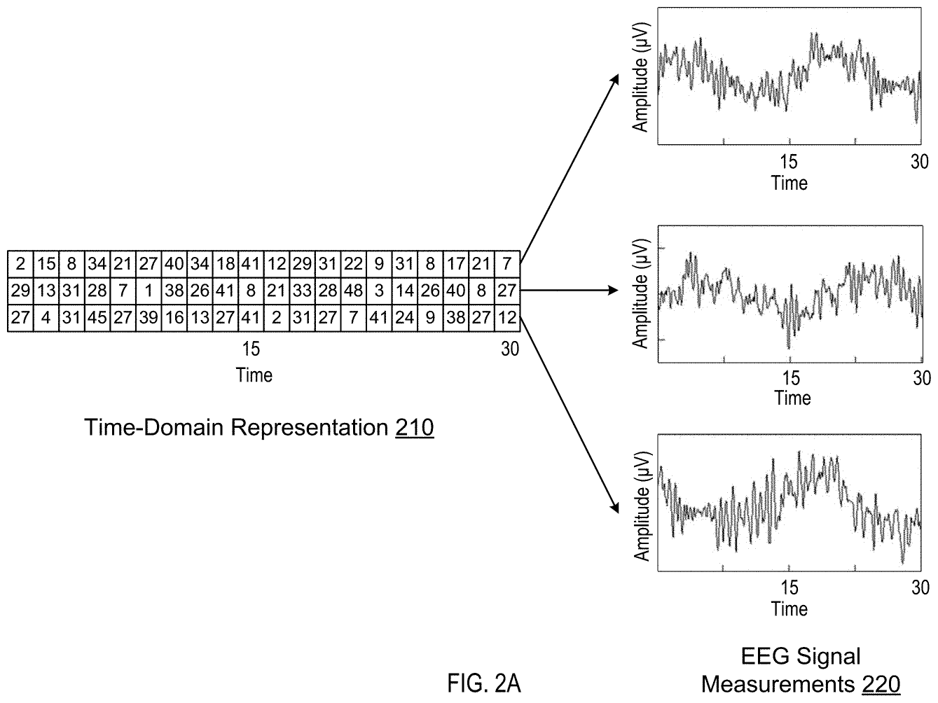

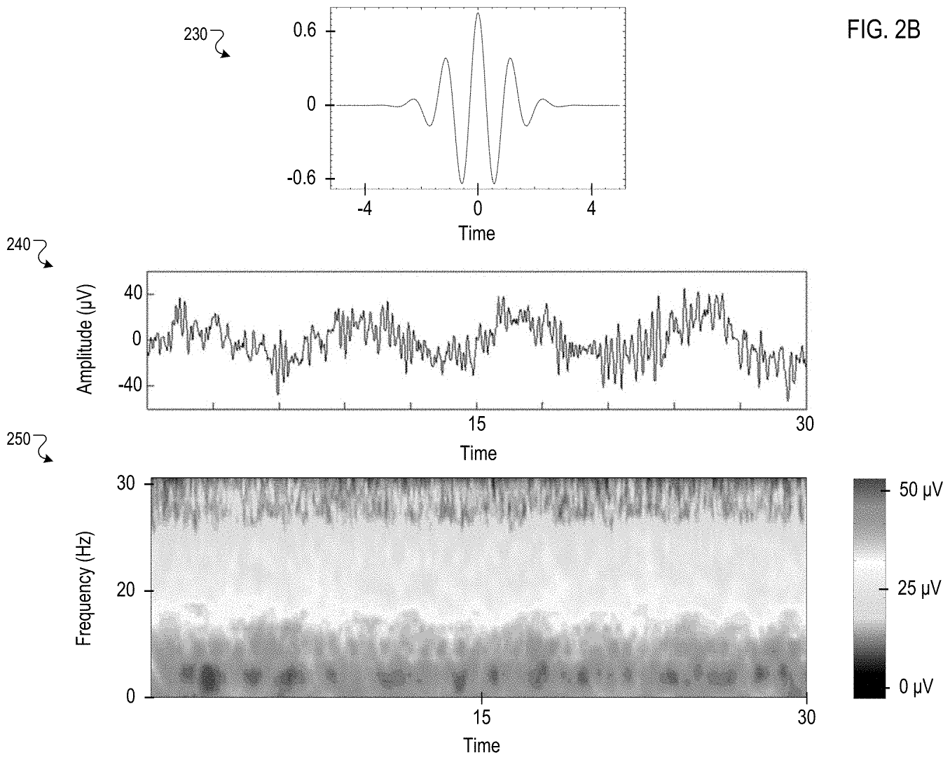

In this specification, a time-domain representation of N EEG signal measurements is a representation of the EEG signal measurements that includes two or more dimensions, where a first dimension corresponds to time and a second dimension corresponds to the N EEG signal measurements. A value corresponding to a particular time in the first dimension and a particular EEG signal measurement in the second dimension identifies a value of the particular EEG signal measurements at the particular time. In some implementations, a third dimension of the time-domain representation corresponds to a channel of the EEG signal measurements. That is, a value corresponding to a particular time in the first dimension, a particular EEG signal measurement in the second dimension, and a particular channel in the third dimension identifies a value of the particular channel of the particular EEG signal measurement at the particular time. Time-domain representations of EEG signal measurements are discussed in more detail below with respect to FIG. 2A.

The frequency-domain representation subsystem 120 obtains the EEG signal measurements 102a-n and processes the EEG signal measurements 102a-n to generate a combined frequency-domain representation 122 of the EEG signal measurements 102a-n. The combined frequency-domain representation 122 of the EEG signal measurements 102a-n is a combination of respective frequency-domain representations of each EEG signal measurement 102a-n.

A frequency-domain representation of an EEG signal measurement represents, for each time point in the EEG signal measurement, the power spectrum of frequencies of the EEG signal measurement at the time point. A first dimension of the two-dimensional representation is time and includes each time point in the sequence of time points of the EEG signal measurements, and a second dimension of the two-dimensional representation is frequency. The second dimension includes multiple frequencies or multiple ranges of frequencies, i.e., multiple different intervals of consecutive frequencies. In this specification, the selection of the multiple frequencies or multiple ranges of frequencies corresponding to a particular frequency-domain representation is referred to as the “frequency range” corresponding to the frequency-domain representation.

That is, the frequency-domain representation of an EEG signal measurement characterizes the relative power of multiple different frequencies through time. For instance, each element in the frequency-domain representation characterizes the power of the EEG signal measurement of the corresponding frequency at the corresponding time. In implementations in which the EEG signal measurement includes multiple channels each corresponding to a different EEG sensor placed on the scalp of the user, the frequency-domain representation can also include multiple channels, where each channel is a frequency-domain representation of the measurement corresponding to the respective EEG sensor placed on the scalp of the user.

In some implementations, the frequency-domain representation subsystem 120 can generate, for each EEG signal measurement 102a-n, the respective frequency-domain representation by determining the Fourier transform of the EEG signal measurement, e.g., by processing the EEG signal measurement using a fast Fourier Transform (FFT) algorithm.

In some other implementations, the frequency-domain representation subsystem 120 can generate, for each EEG signal measurement 102a-n, the respective frequency-domain representation using a wavelet transform. That is, the subsystem 120 can process the EEG signal measurement using multiple versions of a wavelet each corresponding to a different frequency or frequency range. By performing one-dimensional convolution on the EEG signal measurement using a version of the wavelet corresponding to a particular frequency, the system can determine the power of the particular frequency at each time point in the EEG signal measurement. This process is described in more detail below with respect to FIG. 2B.

After generating the respective frequency-domain representations of each EEG signal measurement 102a-n, the frequency-domain representation subsystem 120 combines the N frequency-domain representations to generate the combined frequency-domain representation 122 that characterizes all of the EEG signal measurements 102a-n. In the implementations in which each individual frequency-domain representation includes multiple channels that each correspond to respective EEG sensors placed on the scalp of the user, the subsystem 120 can combine, for each channel, the channel in each frequency-domain representation to generate a corresponding channel in the combined frequency-domain representation 122.

In some implementations, the signal combination subsystem 120 determines a mean or median of the N frequency-domain representation. That is, for each time point and for each frequency in the frequency-domain representation, the frequency-domain representation subsystem 120 can determine the value corresponding to the time point and frequency in the combined frequency-domain representation 122 to be the mean or median of the corresponding values in the N individual frequency-domain representations. In some implementations, when determining the mean or median corresponding to a time point and frequency, the subsystem 120 can discard one or more outlier values corresponding to respective frequency-domain representation.

In some other implementations, the frequency-domain representation subsystem 120 can process each individual frequency-domain representation using a neural network to generate the combined frequency-domain representation 122. As a particular example, the subsystem 120 can process each frequency-domain representation using an attention-based transformer neural network.

The transfer-learned neural network 130 can obtain the time-domain representation 112 and the combined frequency-domain representation 122, and process the representations to generate the mental health prediction 132.

In some implementations, the transfer-learned neural network 130 processes each input, including time-domain representation 112 and the frequency-domain representation 122, using the same subnetwork. For example, the transfer-learned neural network 130 can combine the time-domain representation 112 and the frequency-domain representation 122 into a single network input, e.g., a single image where the time-domain representation 112 is a first channel of the image and the frequency-domain representation 122 is a second channel of the image. In some such implementations, the transfer-learned neural network 130 can generate additional channels of the image. As a particular example, a third channel can be the sum, difference, maximum, or minimum of the time-domain representation 112 and the frequency-domain representation 122. The transfer-learned neural network 130 can then process the combined network input to generate the mental health prediction 132, e.g., using one or more convolutional neural network layers.

In some other implementations, the transfer-learned neural network 130 can process each input, including the time-domain representation 112 and the frequency-domain representation 122 using respective different subnetworks, and generate a respective subnetwork output for each input. The transfer-learned neural network 130 can then combine the multiple subnetwork outputs to generate the mental health prediction 132. For example, the transfer-learned neural network 130 can determine a mean of the multiple subnetwork outputs. As another example, the transfer-learned neural network can use a voting algorithm to generate the mental health prediction, e.g., determine the subnetwork output that is occurs most frequency among the multiple subnetwork outputs and determine the mental health prediction 132 according to the most-frequent subnetwork output.

In this specification, a neural network is “transfer-learned” if at least some of the parameters of the neural network have been trained, at least in part, by processing training examples to generate respective network outputs related to a different machine learning task. Often, a neural network can learn, when being trained to perform a first machine learning task, to extract information that is useful for a second machine learning task. In this case, the parameters of the transfer-learned neural network 130 can be trained according to a different image processing task, e.g., object detection or semantic segmentation. Thus, the parameters of the network 130 can learn to extract information from images that is useful for executing the different image processing task, and thus to extract useful information from the time-domain representation 112 and the frequency-domain representation 122.

In other words, a first training system can train an initial neural network to execute the different image processing task. A second training system can then obtain the trained parameters of the initial neural network, and configure the transfer-learned neural network 130 using the trained parameters of the initial neural network. In some implementations, the first training system and the second training system are the same system.

For example, the second training system can remove one or more layers from the initial neural network to generate the transfer-learned neural network 130, e.g., the final neural network layer. As another example, the training system can fine-tune the values of the parameters of the initial neural network to generate the final values of the parameters of the transfer-learned neural network 130, i.e., process a training data set of time-domain or frequency-domain EEG representations to update the values of the parameters of the pretrained neural network.

In some implementations, the EEG processing system 100 can have only the time-domain representation subsystem 110; that is, the transfer-learned neural network 130 can be configured to process time-domain representations, and not frequency-domain representations, to generate mental health predictions. In some other implementations, the EEG processing system 100 can have only the frequency-domain representation subsystem 120; that is, the transfer-learned neural network 130 can be configured to process frequency-domain representation, and not time-domain representation, to generate mental health predictions.

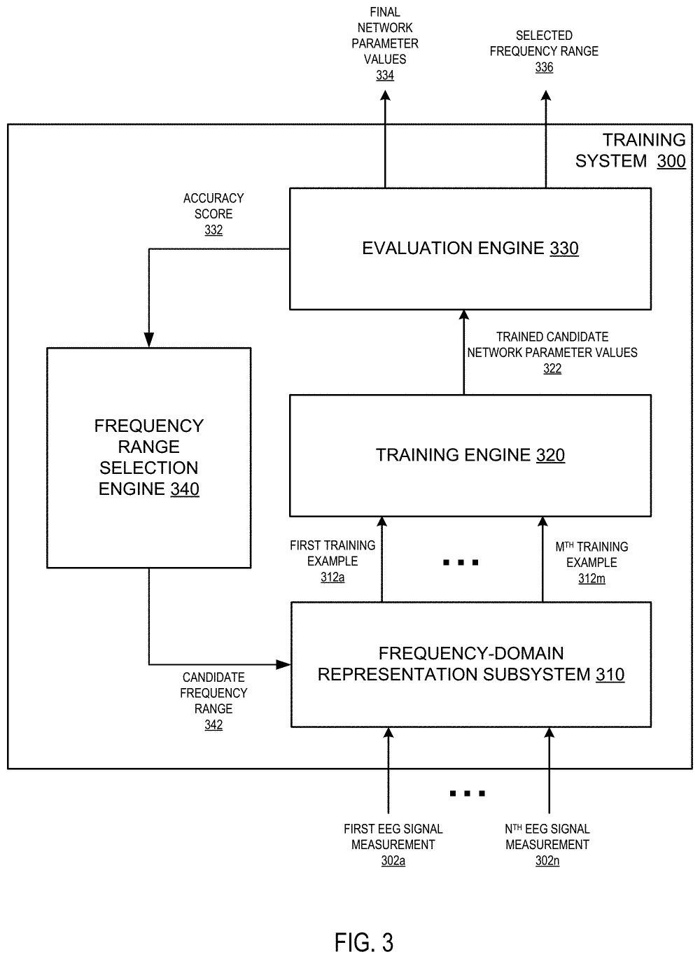

In some implementations, the EEG processing system 100 can include multiple different frequency-domain representation subsystems that each generate different combined frequency-domain representations corresponding to respective frequency ranges. That is, each frequency-domain representation represents a different selection of frequencies or frequency ranges. In these implementations, the transfer-learned neural network 130 can obtain each of the frequency-domain representations and process each frequency-domain representation, optionally with the time-domain representation, to generate the mental health prediction 132. An example process for selecting one or more of the frequency ranges is discussed below with respect to FIG. 3.

FIG. 2A is an illustration of an example time-domain representation 210 of EEG signal measurements 220. The time-domain representation 210 can be generated by a system using the EEG signal measurements 220, e.g., by the time-domain representation subsystem 110 depicted in FIG. 1. Each of the EEG signal measurements 220 correspond to respective different EEG trials of the same user.

The time-domain representation 210 has two dimensions: a dimension corresponding to time and a dimension corresponding to the multiple EEG signal measurements 220. As depicted in FIG. 2A, each EEG signal measurement has a corresponding row of the time-domain representation 210. In some other implementations, each EEG signal measurement has a corresponding column of the time-domain representation 210. For each row of the time-domain representation 210 corresponding to a particular EEG signal measurement, each element of the row corresponds to a time point in the EEG signal measurement. The value of the element is equal to the value of the EEG signal measurement at the time point.

In some implementations, the time-domain representation 210 can have a third dimension corresponding to different EEG sensors. That is, the time-domain representation 210 can have multiple channels, where each channel is generated according to the EEG signal measurements captured by a respective EEG sensor on the scalp of the user. For a given row in the time-domain representation 210, each channel of the row corresponds to the same EEG trial but a different respective EEG sensor used during the EEG trial.

In some implementations, each row of the time-domain representation 210 corresponds to multiple different EEG signal measurements, instead of a single EEG signal measurement. For example, the system can determine an average of multiple different EEG signal measurements, e.g., 5 or 10 EEG signal measurements, and generate the row of the time-domain representation 210 according to the average. That is, for each element of the row, the value of the element is equal to the average of the values of the multiple EEG signal measurements at the corresponding time point.

After generating the time-domain representation 210, the system can process the time-domain representation, e.g., using a convolutional neural network, to generate a mental health prediction for the user.

FIG. 2B is an illustration of an example frequency-domain representation 250 of an EEG signal measurement 240. A system, e.g., the frequency-domain EEG representation subsystem 120 depicted in FIG. 1, can process the EEG signal measurement 240 using a wavelet transform to generate the two-dimensional frequency-domain representation. The two-dimensional frequency-domain representation includes, for each time point in the EEG signal measurement 240, a respective frequency power value for each of multiple frequencies or frequency ranges, and is represented by the spectrogram 250. In this specification, a spectrogram is a visual representation of a two-dimensional frequency-domain EEG representation, where different frequency power values are represented by different pixel intensities or colors.

As a particular example, the system can process the EEG signal measurement 240 using multiple different Morlet wavelets that each correspond to a different frequency. A Morlet wavelet, e.g., the Morlet wavelet 230 illustrated in FIG. 2B is a wavelet composed of a complex exponential multiplied by a Gaussian window. The shape of a Morlet wavelet is defined by a single parameter σ, often called the “number of cycles” of the Morlet wavelet.

For a single parent Morlet wavelet having parameter σ, the system can determine multiple children wavelets having respective frequencies. The system can then convolve each child wavelet along the time dimension of the EEG signal measurement 240 to determine the power of the frequency corresponding to the child wavelet at each time point.

Thus, to generate the frequency-domain representation of the EEG signal measurement 240, the system can determine i) the number of cycles σ, ii) the frequencies of interest, and ii) a sampling rate. In some implementations, the system can select a different parameter σ for each frequency. In general, a lower number of cycles increases temporal domain precision and a higher number of cycles increases the frequency domain precision of the resulting frequency-domain representation.

In some implementations, the system can separately process each of multiple channels of the EEG signal measurement 240 using the Morlet wavelets, where each channel corresponds to a different EEG sensor on the scalp of the user. Thus, the system can generate a respective spectrogram 250 corresponding to each EEG sensor on the scalp of the user.

FIG. 3 is a diagram of an example training system 300. The training system 300 is an example of a system implemented as computer programs on one or more computers in one or more locations, in which the systems, components, and techniques described below can be implemented.

The training system 300 is configured to train a neural network to process an input that includes a frequency-domain representation of EEG signal measurements of a user and to generate a mental health prediction for the user. In particular, the training system 300 is configured to determine, during training of the neural network, the optimal frequency range for the frequency-domain representation. That is, some frequency ranges can be more informative than others for the task of generating a mental health prediction; the training system 300 is configured to automatically learn the frequency range that yields the most accurate mental health predictions.

The training system 300 includes a frequency-domain representation subsystem 310, a training engine 320, an evaluation engine 330, and a frequency range selection engine 340.

The frequency-domain representation subsystem 310 is configured to obtain NEEG signal measurements 302a-n corresponding to respective EEG trials of the same user, and to process the EEG signal measurements 302a-n to generate M training examples 312a-m. Each training example 312a-m includes a frequency-domain representation of EEG data. Each training example 312a-m can also include a “ground-truth” output, i.e., a mental health prediction that the neural network should generate after processing the training example.

The training system 300 can train the neural network across multiple training time points. At the first training time point, the frequency-domain representation subsystem 310 can generate the frequency-domain representation of EEG data according to a predetermined first frequency range. At each subsequent training time point, the frequency-domain representation subsystem 310 can generate the frequency-domain representation of EEG data according to a candidate frequency range 342 determined by the frequency range selection engine 340. As described above, the first frequency range is a selection of one or more frequencies or ranges of frequencies that are represented by the frequency-domain representations.

In some implementations, each of the M training examples 312a-m corresponds to a single EEG signal measurement 302a-n. That is, the frequency-domain representation subsystem 310 can process a single EEG signal measurement 302a-n to generate a training example 312a-m. In some other implementations, each of the M training examples 312a-m correspond to multiple EEG signal measurements 302a-n. That is, as described above, the frequency-domain representation subsystem 310 can process multiple EEG signal measurements 302a-n to generate respective individual frequency-domain representations, and then combine the multiple individual frequency-domain representations to generate a training example 312a-m.

At each training time point, the training engine 320 is configured to process the training examples 312a-m to generate a first set of trained candidate network parameter values 322 of the neural network. The trained candidate network parameter values 322 include values for each parameter of the neural network. For example, the training engine 320 can process one or more training examples 312a-m to generate a “predicted” mental health prediction. The training engine 320 can then determine an error between the “predicted” mental health prediction and the “ground-truth” mental health prediction, and determine an update to the parameters of the neural network according to the error, e.g., using backpropagation.

In some implementations, the training engine 320 updates the value of each parameter of the neural network during training. In some other implementations, the training engine 320 can update the values of a subset of the parameters of the neural network during training, and “freeze” the other parameters of the neural network. That is, the other parameters have the same value in each set of trained network parameter values 322 of the neural network.

At each training time point, the evaluation engine 330 is configured to obtain the first set of trained candidate network parameter values 322, and determine an accuracy score 332 of the first set of trained candidate network parameter values 322. The accuracy score 332 represents an accuracy of the neural network at generating mental health predictions from frequency-domain representations For example, the evaluation engine 330 can process one or more testing EEG examples using the neural network to generate respective “predicted” mental health predictions, and determine an error in the “predicted” mental health predictions. Each testing example can include one or more frequency-domain representations of EEG data of a user, and a “ground-truth” mental health prediction of the user.

The frequency range selection engine 340 is configured to determine, at each training time point, a next candidate frequency range 342 to evaluate. Each candidate frequency range 342 can include a set of frequencies or ranges of frequencies that correspond to rows of the frequency-domain representations, as described above. In some implementations, the frequency range selection engine 340 obtains a predetermined list of candidate frequency ranges to evaluate, and at each training time step selects the next frequency range from the list. For example, the frequency range selection engine 340 can obtain a list of candidate frequency ranges that includes a respective candidate frequency range 342 corresponding to the delta frequency band (typically 1-3 Hz), theta frequency band (typically 3-8 Hz), alpha frequency band (typically 8-13 Hz), beta frequency band (typically 13-38 Hz), and gamma frequency band (typically 38-42 Hz).

In some other implementations, the frequency range selection engine 340 can select the next candidate frequency range 342 according to the accuracy of the previously-evaluated candidate frequency ranges. That is, the frequency range selection engine 340 can, at each training time point, obtain the accuracy score 332 and determine the next candidate frequency range 342 according to the accuracy score 332. For example, the frequency range selection engine 340 can use a hyperparameter optimization algorithm to select the next candidate frequency range 342; that is, the frequency range selection engine 340 can treat the frequency range of the frequency-domain representations of EEG data as a hyperparameter of the neural network. As a particular example, the frequency range selection engine 340 can use a random-search algorithm, a Bayesian optimization algorithm, a gradient-based optimization algorithm, or an evolutionary optimization algorithm.

In some implementations, the frequency range selection engine 340 can select values for multiple hyperparameters of the neural network, including selecting the next candidate frequency range 342, at the same time. For example, the frequency range selection engine 340 can use a grid search algorithm to search the space of multiple hyperparameters.

After the frequency range selection engine 340 determines the next candidate frequency range 342, the training system 300 can repeat the process of evaluating the candidate frequency range 342. That is, the frequency-domain representation subsystem 310 can generate training examples according to the candidate frequency range 342, the training engine 320 can train a version of the neural network using the generated training examples, and the evaluation engine 330 can evaluate the accuracy of the trained version of the neural network. In some implementations, the training engine 320 uses the same M training examples 312a-m to generate training examples at each training time point. In some other implementations, the training engine 320 using a different set of training examples 312a-m to generate training examples at each training time point.

After the final training time point, the training system 300 can then determine the most accurate set of candidate network parameters, and select a final frequency range corresponding to the most accurate candidate set of network parameters. That is, the training system 300 can determine the set of trained candidate network parameter values 322, trained during a respective training time point, that has the highest corresponding accuracy score 332. The training system 300 can then output the selected final network parameter values 334 and the frequency range 336 corresponding to the final network parameter values 334.

In some implementations, the training system 300 can provide the final network parameter values 334 and the selected frequency range 336 to an inference system that processes EEG data to generate mental health predictions. In some other implementations, the training system 300 can provide the final network parameter values 334 and the selected frequency range 336 to another system that will further train, i.e., “fine-tune,” the network parameter values 334 according to the frequency range 336.

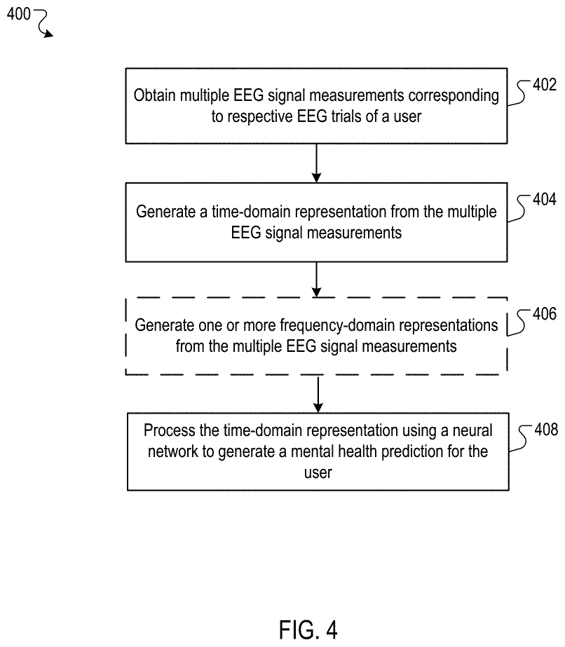

FIG. 4 is a flow diagram of an example process 400 for processing one or more EEG signal measurements to generate a mental health prediction for a user. For convenience, the process 400 will be described as being performed by a system of one or more computers located in one or more locations. For example, an EEG processing system, e.g., the EEG processing system 100 depicted in FIG. 1, appropriately programmed in accordance with this specification, can perform the process 400.

The system obtains multiple EEG signal measurements corresponding to respective EEG trials of a user (step 402). In some implementations, each EEG signal measurement was captured from the brain of the user in response to the same particular prompt of the same EEG task. In some implementations, each EEG signal measurement has multiple channels each corresponding to a respective EEG sensor.

The system processes the multiple EEG signal measurements to generate a time-domain representation of the EEG signal measurements (step 404). As described above with reference to FIG. 2A, the time-domain representation includes a first dimension corresponding to the multiple EEG signal measurements and a second dimension corresponding to time. In implementations in which each EEG signal measurement has multiple channels, the time-domain representation can have a third dimension corresponding to the multiple channels.

In some implementations, the time-domain representation includes multiple rows that each correspond to a different set of one or more EEG signal measurements. Each row can correspond to a single EEG signal measurement or multiple EEG signal measurements. As a particular example, each row can characterize an average EEG signal computed from multiple EEG signal measurements. In some other implementations, the time-domain representation includes multiple columns that each correspond to a different set of one or more EEG signal measurements.

In some implementations, the time-domain representation includes multiple two-dimensional channels that each correspond to a respective EEG sensor.

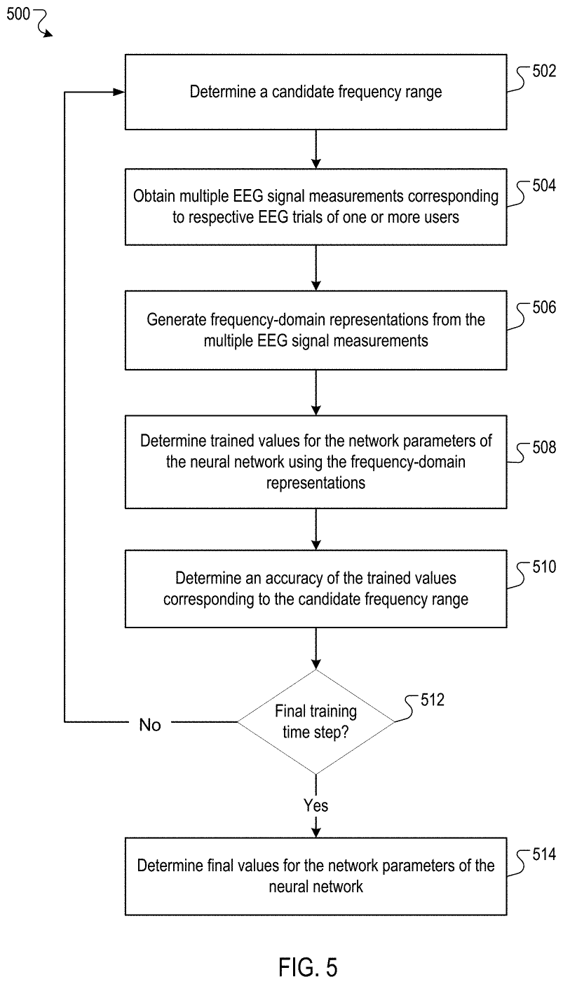

Optionally, the system processes the multiple EEG signal measurements to generate one or more frequency-domain representations of the EEG signal measurements (step 406). Each frequency-domain representation corresponds to a respective different frequency range. In some implementations, the one or more frequency ranges are determined according to a training process described below with respect to FIG. 5.

The system processes the time-domain representation using a neural network to generate a mental health prediction for the user, according to final values of multiple network parameters of the neural network (step 408). For example, as described above, the mental health prediction can characterize a likelihood that the user has a particular mental health disorder, e.g., a value between 0 and 1 representing a probability that the user has the particular mental health disorder.

In some implementations in which the system generates one or more frequency-domain representations, the system provides the one or more frequency-domain representations as input to the same neural network. For example, the input to the neural network can include an image, where a first channel of the image is the time-domain representation and one or more second channels of the image are the one or more frequency-domain representations.

In some other implementations in which the system generates one or more frequency-domain representations, the system can process the one or more frequency-domain representations with a second neural network to generate a second mental health prediction. The system can then process the mental health prediction corresponding to the time-domain representation and the second mental health prediction corresponding to the one or more frequency-domain representations to generate a final mental health prediction.

In some other implementations in which the system generates multiple frequency-domain representations, the system can process each frequency-domain representation using a respective different third neural network to generate a respective different third mental health prediction. The system can then process the mental health prediction corresponding to the time-domain representation and the multiple third mental health predictions corresponding to respective frequency-domain representations to generate a final mental health prediction. In some implementations, processing multiple different mental health predictions to generate a final mental health prediction includes determining the final mental health prediction to be equal to the average of the multiple different mental health predictions. In some other implementations, processing multiple different mental health predictions to generate a final mental health prediction includes executing a voting algorithm, e.g., determining the final mental health prediction to be equal to the mental health prediction that occurs most frequently in the multiple mental health predictions.

In some implementations, the neural network has been trained using transfer learning. That is, the final values of the network parameters having been determined by a transfer learning process wherein the neural network is initially trained to perform an image processing task by using a first training data set of multiple training images to determine initial values for the plurality of network parameters, and the final values for the network parameters are subsequently determined according to the initial values of the network parameters for performing EEG analysis. In some implementations, the system determines the initial values; in some other implementations, the system obtains the initial values from a different system.

In other words, the neural network, according to the initial values of the network parameters, is configured to process an input that includes an image to generate a corresponding output, e.g., a classification output, a regression output, or a combination thereof.

As a particular example, the neural network can be configured to process an image to generate a classification output that includes a respective score corresponding to each of multiple categories. The score for a category indicates a likelihood that the image belongs to the category. In some cases, the categories may be classes of objects (e.g., dog, cat, person, and the like), and the image may belong to a category if it depicts an object included in the object class corresponding to the category. In some cases, the categories may represent global image properties (e.g., whether the image depicts a scene in the day or at night, or whether the image depicts a scene in the summer or the winter), and the image may belong to the category if it has the global property corresponding to the category.

As another particular example, the neural network can be configured to process an image to generate a pixel-level classification output that includes, for each pixel, a respective score corresponding to each of multiple categories. For a given pixel, the score for a category indicates a likelihood that pixel belongs to the category. In some cases, the categories may be classes of objects, and a pixel may belong to a category if it is part of a depiction of an object included in the object class corresponding to the category. That is, the pixel-level classification output may be semantic segmentation output. As a particular example, the neural network may be an edge detector neural network configured to predict one or more pixels of an image that represent edges of objects depicted in the image.

As another particular example, the neural network can be configured to process an image to generate a regression output that estimates one or more continuous variables (i.e., that can assume infinitely many possible numerical values) that characterize the image. In a particular example, the regression output may estimate the coordinates of bounding boxes that enclose respective objects depicted in the image. The coordinates of a bounding box may be defined by (x, y) coordinates of the vertices of the bounding box.

In some implementations, determining the final values for the network parameters includes fine-tuning the initial values using a second training data set that includes multiple training time-domain representations of EEG signal measurements. In some other implementations, the initial values are the same as the final values.

In some implementations, determining the final values for the network parameters from the initial values includes removing a subset of the network parameters; that is, the set of initial values can include values corresponding to parameters that are not represented in the set of final values. For example, the system might remove one or more neural network layers from the neural network.

In some implementations, determining the final values for the network parameters from the initial values includes adding additional network parameters; that is, the set of final values can include values corresponding to parameters that are not represented in the set of initial values. For example, the system might add one or more additional neural network layers to the neural network, and determine the values for the parameters of the additional neural network layers using the second training data set.

FIG. 5 is a flow diagram of an example process 500 for determining the optimal frequency range of frequency-domain representations for generating mental health predictions for users. After the optimal frequency range is determined, the frequency-domain representations will be processed by a neural network that has multiple network parameters to generate the mental health predictions. For convenience, the process 500 will be described as being performed by a system of one or more computers located in one or more locations. For example, a training system 300 depicted in FIG. 3, appropriately programmed in accordance with this specification, can perform the process 500.

The system can repeat step 502-510 of the process 500 for each of multiple training time steps.

The system determines a candidate frequency range (step 502). At the first training time step, the candidate frequency range can be a predetermined frequency range. At each subsequent training time steps, the system can determine candidate frequency range using accuracy scores characterizing the performance of neural networks trained in previous training time steps.

The system obtains multiple EEG signal measurements corresponding to respective EEG trials of one or more users (step 504). That is, the EEG signal measurements can include data captured from more than one user. In some implementations the system uses the same set of EEG signal measurements during each training time step.

The system generates frequency-domain representations from the multiple EEG signal measurements (step 506). In particular, the system generates the frequency-domain representations according to the current candidate frequency range; that is, the system can determine the amplitude of each identified frequency in the candidate frequency range at each time point.

In some implementations, the system processes multiple different EEG signal measurements to generate each frequency-domain representation; in some other implementations, the system processes a single EEG signal measurement for each frequency-domain representation.

The system processes the generated frequency-domain representations to determine trained values for the network parameters of the neural network (step 508). For example, the system can process one or more of the frequency-domain representations to generate a “training” mental health prediction, and obtain a “ground-truth” mental health prediction. The system can then backpropagate an error between the “training” prediction and the “ground-truth” prediction through the neural network to determine an update to the network parameters.

In some implementations, a strict subset of the network parameters are trained while the remaining network parameters are held constant during training.

The system determines an accuracy of the trained values of the network parameters of the neural network corresponding to the candidate frequency range (step 510). For example, the system can process one or more “testing” frequency-domain representations, e.g., through cross-validation on the frequency-domain representations generated in step 506, to generate “testing” mental health predictions, and then determine an accuracy of the “testing” mental health predictions.

The system determines if the current training time step is the final training time step (step 512). In some implementations, the system executes a predetermined number of training time steps. In some other implementations, the system determines whether the current training time step is the final training time step according to one or more criteria. As a particular example, the system might determine that the current training time step is the final training time step if the accuracy scores determined in step 510 have stopped improving, e.g., if an increase in accuracy scores across time steps has dropped below a predetermined threshold.

If the system determines that the current training time step is not the final training time step, the system returns to step 502 and begins a new training time step by determining the next candidate frequency range.

If the system determines that the current training time step is the final training time step, then the system determines a final frequency range and final values for the network parameters of the neural network (step 514). The system can determine the final frequency range and final values to be the frequency range and trained values corresponding to the highest accuracy score.

In some implementations, the system can select multiple final frequency values, e.g., the N frequency values with the highest corresponding accuracy scores. The system can then output the N sets of trained parameter values corresponding to the selected final frequency ranges, e.g., as the parameter values for N respective subnetworks of an ensemble model. That is, the system can provide the N final frequency ranges and trained parameter values to a downstream model that is configured to process frequency-domain representations to generate N respective network outputs. The downstream model can then combine the N network outputs to generate a final mental health prediction.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

In addition to the embodiments described above, the following embodiments are also innovative:

Embodiment 1 is a method comprising: obtaining a plurality of EEG signal measurements corresponding to respective EEG trials of a user; generating a time-domain representation from the plurality of EEG signal measurements, wherein the time-domain representation comprises a plurality of rows, and wherein each row corresponds to a different set of one or more EEG signal measurements; applying the time-domain representation as input to a neural network having a plurality of network parameters, final values of the network parameters having been determined by a transfer learning process wherein the neural network is initially trained to perform an image processing task by using a first training data set comprising a plurality of training images to determine initial values for the plurality of network parameters, and the neural network is subsequently trained to perform EEG analysis by using a second training data set to determine the final values for the plurality of network parameters from the initial values of the plurality of network parameters; and obtaining, from the neural network, a mental health prediction for the user.

Embodiment 2 is the method of embodiment 1, wherein the second training data set comprises a plurality of training time-domain representations of EEG signal measurements, each training time-domain representation comprising a plurality of rows each corresponding to a different set of one or more EEG signal measurements, and wherein the final values for the plurality of network parameters are determined by updating the initial values of the plurality of network parameters based on the second training data set.

Embodiment 3 is the method of any one of embodiments 1 or 2, further comprising: generating a frequency-domain representation from the plurality of EEG signal measurements; and applying the frequency-domain representation as an input to the neural network to generate the mental health prediction for the user.

Embodiment 4 is the method of embodiment 3, wherein the input to the neural network comprises an image, wherein a first channel of the image is the time-domain representation and a second channel of the image is the frequency-domain representation.

Embodiment 5 is the method of any one of embodiments 1-4, further comprising: generating a frequency-domain representation from the plurality of EEG signal measurements; applying the frequency-domain representation as an input to a second neural network having a plurality of second network parameters to generate a second mental health prediction for the user, wherein the second neural network has been trained using transfer learning; and processing i) the mental health prediction generated by the neural network and ii) the second mental health prediction generated by the second neural network to generate a final mental health prediction for the user.

Embodiment 6 is the method of any one of embodiments 1-5, further comprising: determining a plurality of different frequency ranges; for each of the plurality of frequency ranges: processing the plurality of EEG signal measurements to generate a frequency-domain representation corresponding to the frequency range; and processing the frequency-domain representation using a third neural network corresponding to the frequency range and having a plurality of third network parameters to generate a respective third mental health prediction for the user, wherein the third neural network has been trained using transfer learning; and processing i) the mental health prediction generated by the neural network and ii) the plurality of third mental health predictions generated by respective third neural networks to generate a final mental health prediction for the user.

Embodiment 7 is the method of embodiment 6, wherein processing i) the mental health prediction generated by the neural network and ii) the plurality of third mental health predictions generated by respective third neural networks to generate a final mental health prediction for the user comprises one or more of: determining an average of i) the mental health prediction generated by the neural network and ii) the plurality of third mental health predictions generated by respective third neural networks; or processing i) the mental health prediction generated by the neural network and ii) the plurality of third mental health predictions generated by respective third neural networks according to a voting algorithm to generate the final mental health prediction.

Embodiment 8 is the method of any one of embodiments 1-7, wherein each row of the time-domain representation characterizes an average EEG signal measurement generated from a different set of a plurality of EEG signal measurements.

Embodiment 9 is the method of any one of embodiments 1-8, wherein the time-domain representation comprises a plurality of two-dimensional channels each corresponding to a different EEG sensor.

Embodiment 10 is the method of any one of embodiments 1-9, wherein the mental health prediction characterizes a likelihood that the user has a particular mental health disorder.

Embodiment 11 is a neural network training method comprising: obtaining a neural network having a plurality of network parameters; training the neural network to perform an image processing task by determining initial values for the plurality of network parameters using a first training data set comprising a plurality of training images; and training the neural network to perform EEG analysis by using a second training data set to determine final values for the plurality of network parameters from the initial values of the plurality of network parameters.

Embodiment 12 is the method of embodiment 11, wherein the neural network is configured to process a network input generated from EEG data of a user and to generate a network output characterizing a mental health prediction for the user.

Embodiment 13 is the method of any one of embodiments 11 or 12, wherein the second training data set comprises a plurality of training time-domain representations of EEG signal measurements, each training time-domain representation comprising a plurality of rows each corresponding to a different set of one or more EEG signal measurements, and wherein the final values for the plurality of network parameters are determined by updating the initial values of the plurality of network parameters based on the second training data set.

Embodiment 14 is the method of any one of embodiments 11-13, wherein the neural network is configured to process a network input comprising a frequency-domain representation of EEG data of a user, and wherein training the neural network to perform EEG analysis comprises determining a frequency range for the frequency-domain representation.

Embodiment 15 is the method of embodiment 14, wherein determining a frequency range for the frequency-domain representation comprises: at each of a plurality of training time points: determining a candidate frequency range; generating a plurality of training examples comprising frequency-domain representations of EEG data according to the candidate frequency range; training the neural network using the plurality of generated training examples to determine candidate values for the plurality of network parameters; and determining an accuracy score for the neural network characterizing an accuracy of the trained candidate values; and selecting, from the plurality of candidate frequency ranges, a final frequency range according to the determined accuracy scores.

Embodiment 16 is the method of embodiment 15, wherein determining a candidate frequency range at each of the plurality of training time points comprises determining the candidate frequency range according to one or more of: a random-search algorithm, a Bayesian optimization algorithm, a gradient-based optimization algorithm, or

an evolutionary optimization algorithm.

Embodiment 17 is a system comprising: one or more computers and one or more storage devices storing instructions that are operable, when executed by the one or more computers, to cause the one or more computers to perform the method of any one of embodiments 1 to 16.

Embodiment 18 is one or more non-transitory computer storage media encoded with a computer program, the program comprising instructions that are operable, when executed by data processing apparatus, to cause the data processing apparatus to perform the method of any one of embodiments 1 to 16.

Embodiment 19 is a neural network training method comprising: obtaining a neural network having a plurality of network parameters; training the neural network to perform an image processing task by determining initial values for the plurality of network parameters using a first training data set comprising a plurality of training images; and training the neural network to perform EEG analysis by using a second training data set to determine final values for the plurality of network parameters from the initial values of the plurality of network parameters.

Embodiment 20 is the method of embodiment 19, wherein the neural network is configured to process a network input generated from EEG data of a user and to generate a network output characterizing a mental health prediction for the user.

Embodiment 21 is the method of any one of embodiments 19 or 20, wherein the second training data set comprises a plurality of training time-domain representations of EEG signal measurements, each training time-domain representation comprising a plurality of rows each corresponding to a different set of one or more EEG signal measurements, and wherein the final values for the plurality of network parameters are determined by updating the initial values of the plurality of network parameters based on the second training data set.

Embodiment 22 is the method of and one of the embodiments 19-21, wherein the neural network is configured to process a network input comprising a frequency-domain representation of EEG data of a user, and wherein training the neural network to perform EEG analysis comprises determining a frequency range for the frequency-domain representation.

Embodiment 23 is the method of embodiment 22, wherein determining a frequency range for the frequency-domain representation comprises: at each of a plurality of training time points: determining a candidate frequency range; generating a plurality of training examples comprising frequency-domain representations of EEG data according to the candidate frequency range; training the neural network using the plurality of generated training examples to determine candidate values for the plurality of network parameters; and determining an accuracy score for the neural network characterizing an accuracy of the trained candidate values; and selecting, from the plurality of candidate frequency ranges, a final frequency range according to the determined accuracy scores.

Embodiment 24 is the method of embodiment 23, wherein determining a candidate frequency range at each of the plurality of training time points comprises determining the candidate frequency range according to one or more of: a random-search algorithm, a Bayesian optimization algorithm, a gradient-based optimization algorithm, or an evolutionary optimization algorithm.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
obtaining a plurality of EEG signal measurements corresponding to respective EEG trials of a user;
generating a time-domain representation from the plurality of EEG signal measurements, wherein the time-domain representation comprises a plurality of rows, and wherein each row corresponds to a different set of one or more EEG signal measurements;
applying the time-domain representation as input to a neural network having a plurality of network parameters, final values of the network parameters having been determined by a transfer learning process wherein the neural network is initially trained to perform an image processing task by using a first training data set comprising a plurality of training images to determine initial values for the plurality of network parameters, and the neural network is subsequently trained to perform EEG analysis by using a second training data set to determine the final values for the plurality of network parameters from the initial values of the plurality of network parameters; and
obtaining, from the neural network, a mental health prediction for the user.

2. The method of claim 1, wherein the second training data set comprises a plurality of training time-domain representations of EEG signal measurements, each training time-domain representation comprising a plurality of rows each corresponding to a different set of one or more EEG signal measurements, and wherein the final values for the plurality of network parameters are determined by updating the initial values of the plurality of network parameters based on the second training data set.

3. The method of claim 1, further comprising:
generating a frequency-domain representation from the plurality of EEG signal measurements; and
applying the frequency-domain representation as an input to the neural network to generate the mental health prediction for the user.

4. The method of claim 3, wherein the input to the neural network comprises an image, wherein a first channel of the image is the time-domain representation and a second channel of the image is the frequency-domain representation.

5. The method of claim 1, further comprising:
generating a frequency-domain representation from the plurality of EEG signal measurements;
applying the frequency-domain representation as an input to a second neural network having a plurality of second network parameters to generate a second mental health prediction for the user, wherein the second neural network has been trained using transfer learning; and
processing i) the mental health prediction generated by the neural network and ii) the second mental health prediction generated by the second neural network to generate a final mental health prediction for the user.

6. The method of claim 1, further comprising:
determining a plurality of different frequency ranges;
for each of the plurality of frequency ranges:
processing the plurality of EEG signal measurements to generate a frequency-domain representation corresponding to the frequency range; and
processing the frequency-domain representation using a third neural network corresponding to the frequency range and having a plurality of third network parameters to generate a respective third mental health prediction for the user, wherein the third neural network has been trained using transfer learning; and
processing i) the mental health prediction generated by the neural network and ii) the plurality of third mental health predictions generated by respective third neural networks to generate a final mental health prediction for the user.

7. The method of claim 6, wherein processing i) the mental health prediction generated by the neural network and ii) the plurality of third mental health predictions generated by respective third neural networks to generate a final mental health prediction for the user comprises one or more of:
determining an average of i) the mental health prediction generated by the neural network and ii) the plurality of third mental health predictions generated by respective third neural networks; or
processing i) the mental health prediction generated by the neural network and ii) the plurality of third mental health predictions generated by respective third neural networks according to a voting algorithm to generate the final mental health prediction.

8. The method of claim 1, wherein each row of the time-domain representation characterizes an average EEG signal measurement generated from a different set of a plurality of EEG signal measurements.

9. The method of claim 1, wherein the time-domain representation comprises a plurality of two-dimensional channels each corresponding to a different EEG sensor.

10. The method of claim 1, wherein the mental health prediction characterizes a likelihood that the user has a particular mental health disorder.

11. A system comprising one or more computers and one or more storage devices storing instructions that are operable, when executed by the one or more computers, to cause the one or more computers to perform operations comprising:
obtaining a plurality of EEG signal measurements corresponding to respective EEG trials of a user;
generating a time-domain representation from the plurality of EEG signal measurements, wherein the time-domain representation comprises a plurality of rows, and wherein each row corresponds to a different set of one or more EEG signal measurements;
applying the time-domain representation as input to a neural network having a plurality of network parameters, final values of the network parameters having been determined by a transfer learning process wherein the neural network is initially trained to perform an image processing task by using a first training data set comprising a plurality of training images to determine initial values for the plurality of network parameters, and the neural network is subsequently trained to perform EEG analysis by using a second training data set to determine the final values for the plurality of network parameters from the initial values of the plurality of network parameters; and
obtaining, from the neural network, a mental health prediction for the user.

12. The system of claim 11, wherein the second training data set comprises a plurality of training time-domain representations of EEG signal measurements, each training time-domain representation comprising a plurality of rows each corresponding to a different set of one or more EEG signal measurements, and wherein the final values for the plurality of network parameters are determined by updating the initial values of the plurality of network parameters based on the second training data set.

13. The system of claim 11, wherein the operations further comprise:
generating a frequency-domain representation from the plurality of EEG signal measurements; and
applying the frequency-domain representation as an input to the neural network to generate the mental health prediction for the user.

14. The system of claim 11, wherein the operations further comprise:
generating a frequency-domain representation from the plurality of EEG signal measurements;
applying the frequency-domain representation as an input to a second neural network having a plurality of second network parameters to generate a second mental health prediction for the user, wherein the second neural network has been trained using transfer learning; and
processing i) the mental health prediction generated by the neural network and ii) the second mental health prediction generated by the second neural network to generate a final mental health prediction for the user.

15. The system of claim 11, wherein the operation further comprise:
determining a plurality of different frequency ranges;
for each of the plurality of frequency ranges:
processing the plurality of EEG signal measurements to generate a frequency-domain representation corresponding to the frequency range; and
processing the frequency-domain representation using a third neural network corresponding to the frequency range and having a plurality of third network parameters to generate a respective third mental health prediction for the user, wherein the third neural network has been trained using transfer learning; and
processing i) the mental health prediction generated by the neural network and ii) the plurality of third mental health predictions generated by respective third neural networks to generate a final mental health prediction for the user.

16. One or more non-transitory computer storage media encoded with computer program instructions that when executed by a plurality of computers cause the plurality of computers to perform operations comprising:
obtaining a plurality of EEG signal measurements corresponding to respective EEG trials of a user;
generating a time-domain representation from the plurality of EEG signal measurements, wherein the time-domain representation comprises a plurality of rows, and wherein each row corresponds to a different set of one or more EEG signal measurements;
applying the time-domain representation as input to a neural network having a plurality of network parameters, final values of the network parameters having been determined by a transfer learning process wherein the neural network is initially trained to perform an image processing task by using a first training data set comprising a plurality of training images to determine initial values for the plurality of network parameters, and the neural network is subsequently trained to perform EEG analysis by using a second training data set to determine the final values for the plurality of network parameters from the initial values of the plurality of network parameters; and
obtaining, from the neural network, a mental health prediction for the user.

17. The non-transitory computer storage media of claim 16, wherein the second training data set comprises a plurality of training time-domain representations of EEG signal measurements, each training time-domain representation comprising a plurality of rows each corresponding to a different set of one or more EEG signal measurements, and wherein the final values for the plurality of network parameters are determined by updating the initial values of the plurality of network parameters based on the second training data set.

18. The non-transitory computer storage media of claim 16, wherein the operations further comprise:
generating a frequency-domain representation from the plurality of EEG signal measurements; and
applying the frequency-domain representation as an input to the neural network to generate the mental health prediction for the user.

19. The non-transitory computer storage media of claim 16, wherein the operations further comprise:
generating a frequency-domain representation from the plurality of EEG signal measurements;
applying the frequency-domain representation as an input to a second neural network having a plurality of second network parameters to generate a second mental health prediction for the user, wherein the second neural network has been trained using transfer learning; and
processing i) the mental health prediction generated by the neural network and ii) the second mental health prediction generated by the second neural network to generate a final mental health prediction for the user.

20. The non-transitory computer storage media of claim 16, wherein the operation further comprise:
determining a plurality of different frequency ranges;
for each of the plurality of frequency ranges:
processing the plurality of EEG signal measurements to generate a frequency-domain representation corresponding to the frequency range; and
processing the frequency-domain representation using a third neural network corresponding to the frequency range and having a plurality of third network parameters to generate a respective third mental health prediction for the user, wherein the third neural network has been trained using transfer learning; and
processing i) the mental health prediction generated by the neural network and ii) the plurality of third mental health predictions generated by respective third neural networks to generate a final mental health prediction for the user.
